(19) United States
(12) Patent Application Publication
(10) Pub. No.: US 2024/0286990 A1
(43) Pub. Date: Aug. 29, 2024

(54) VINYLATED KETO ESTERS WITH APPLICABILITY IN SIGNAL ENHANCED MAGNETIC RESONANCE IMAGING AND SYNTHESIS THEREOF

(71) Applicant: MAX-PLANCK-GESELLSCHAFT ZUR FÖRDERUNG DER WISSENSCHAFTEN E. V., München (DE)

(72) Inventors: Stefan GLÖGGLER, Göttingen (DE); Sergey KORCHAK, Göttingen (DE); Anil P. JAGTAP, Göttingen (DE); Philip SAUL, Göttingen (DE); Denis MOLL, Göttingen (DE)

(73) Assignee: MAX-PLANCK-GESELLSCHAFT ZUR FÖRDERUNG DER WISSENSCHAFTEN E. V., München (DE)

(21) Appl. No.: 18/571,271

(22) PCT Filed: Jun. 16, 2022
(86) PCT No.: PCT/EP2022/066534

§ 371 Date: Dec. 18, 2023

(30) Foreign Application Priority Data
Jun. 18, 2021 (EP) 21180463.8

(51) Int. Cl. C07C 69/716; C07B 59/00

(52) U.S. Cl. CPC C07C 69/716; C07B 59/001; C07B 2200/05

(57) ABSTRACT

The present invention relates to a vinyl keto ester and intermediates of its synthesis, wherein the vinyl moiety of the vinyl hydroxy ester and of the intermediates is partly or fully deuterated. Furthermore, the present invention relates to two alternative methods of preparing said vinyl keto ester. The first method is a multi-step approach comprising the steps of providing a carboxylic acid that comprises a geminal diol moiety protected by a photolabile protecting group, vinylating said carboxylic acid using vinyl acetate, and cleaving the protecting group by applying UV light. The second method is a one-step approach of reacting a carboxylic acid that comprises an additional carbonyl moiety with acetylene in the presence of a metal catalyst. In both methods, the compounds used may be fully or partly deuterated.

The present invention relates to methods to synthesize vinylated keto esters that are suitable for the preparation of hyperpolarized agents that enhance NMR signals. Furthermore, the present invention relates to vinylated keto esters per se as well as intermediates of their synthesis.

### BACKGROUND OF THE INVENTION

The phenomenon of nuclear magnetic resonance (NMR) and its tomography modality magnetic resonance imaging (MRI) has wide applicability in analytics and clinical diagnostics. NMR is an intrinsically insensitive phenomenon which is why hyperpolarization strategies were devised to improve the sensitivity. Hyperpolarization is thereby a process that enhances NMR signals by several orders of magnitude compared to the normal/thermally polarized signal. In the past years the use of hyperpolarized metabolites was introduced to the field of preclinical and clinical research to study diseases even in patients. The state-of-the-art technique is dynamic nuclear polarization (DNP). For this procedure typically ¹³C and ¹⁵N isotopically enriched molecules are used whereby the most prominent example is ¹³C enriched pyruvate. The molecules are cooled down to cryogenic temperatures at and below 2 K in the presence of radicals inside a dedicated super-conducting high-field magnet. At that low temperature the irradiation with microwaves over tens of minutes to hours leads to the polarization transfer of the highly polarized electron spins of the radicals to the heteronucleus of the desired molecule. Heteronuclei are spins other than protons, ¹H. Protons can also be polarized via the described procedures but are less relevant for preclinical or clinical studies. Subsequently, the hyperpolarized molecules of interest are rapidly thawed and can be used as signal enhanced magnetic resonance contrast agents as they allow to probe metabolic conversion directly in real-time.

Another technique of hyperpolarization is the para-hydrogen induced polarization (PHIP). It is a faster approach than DNP and polarizes metabolites in seconds rather than tens of minutes to hours. In order to signal enhance metabolites suitable precursor molecules are required that can
A) be rapidly reacted with the para-spin isomer of hydrogen,
B) yield high degrees of signal enhancements and
C) can be quickly converted into the molecule of interest.

Using the PHIP approach several metabolites including acetate, lactate and pyruvate where hyperpolarized. However, the obtained polarization of isotopically enriched compounds stays one order of magnitude behind that achievable via DNP. If similar polarization degrees are achieved via PHIP for such metabolic contrast agents this opens up the opportunity to make the production of contrast agents cost-efficient, orders of magnitude faster and widely applicable to health-care institutions because the PHIP technique does not require a dedicated high-field magnet. In contrast, only portable low-field devices are required in which para-hydrogen is reacted via suitable precursors.

So far, NMR experiments have been devised to theoretically deliver optimal results for producing signal-enhanced contrast agents. However, the chemical precursors that promote maximal signal enhancements for important metabolites including lactate and pyruvate do not exist. Literature studies have shown that vinyl carboxylic acids (e.g. vinyl acetate, vinyl lactate and vinyl pyruvate) are the most promising precursor molecules. Although the molecules are known, the isotopic labelling with deuterium has not been achieved so far. To achieve optimal signal enhancements via PHIP not only a ¹³C atom needs to be included into the precursor but ideally at least the vinyl functionality should be deuterated. This is a challenge that has so far not been overcome.

The present invention relates to new chemical procedures that allow to synthesize vinyl esters of keto esters. The most prominent representative of this class of molecules is vinyl pyruvate (α-keto ester). Other molecules of immediate interest are vinyl esters of ketoisocaproate and acetoacetate (keto esters). Although the metabolite's free acid is often more desirable, the uncleaved ester can also be used as contrast agents. In contrast to the known methods and products described above, the present invention allows cost-effective preparation of vinylated keto esters that are suitable for the preparation of hyperpolarized agents.

Based on the above-mentioned state of the art, the objective of the present invention is to provide means and methods to synthesize vinylated keto esters that are suitable for the preparation of hyperpolarized agents that enhance NMR signals. This objective is attained by the subject-matter of the independent claims of the present specification, with further advantageous embodiments described in the dependent claims, examples, figures and general description of this specification.

### SUMMARY OF THE INVENTION

A first aspect of the invention relates to a vinyl keto ester of formula (III),

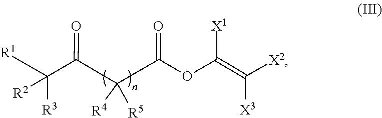

wherein
R¹, R² and R³ are independently of each other selected from H, D, and a fully or partly deuterated alkyl,
each R⁴ and R⁵ are independently of any other R⁴ or R⁵ selected from H and D,
X¹, X² and X³ are independently of each other H or D, wherein at least one of X¹, X² and X³ is D, and
n is an integer between 0 and 16.

A second aspect of the invention relates to a method for preparing a compound suitable for signal enhanced magnetic resonance imaging. The method comprises the steps of
a) providing a compound of formula (I),

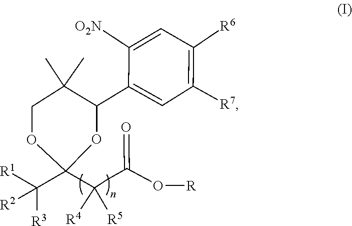

b) vinylation of the compound of formula (I) by using vinyl acetate of formula (II),

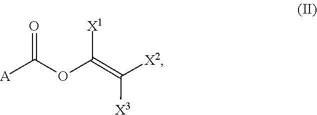

c) applying UV light yielding a vinyl keto ester of formula (III),

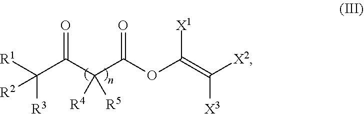

wherein
R is H or D,
R¹, R² and R³ are independently of each other selected from H, D, and a fully or partly deuterated alkyl,
each R⁴ and R⁵ are independently of any other R⁴ or R⁵ selected from H and D,
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
   R^{a} being H or a C₁₋₃-alkyl,
   p being an integer between 0 and 6,
X¹, X² and X³ are independently of each other H or D,
A is —CH₃, —CH₂D, —CHD₂ or -CD₃, and
n is an integer between 0 and 16, particularly between 0 and 6, more particularly between 0 and 3.

A third aspect of the invention relates to a compound of formula (I) or (VI),

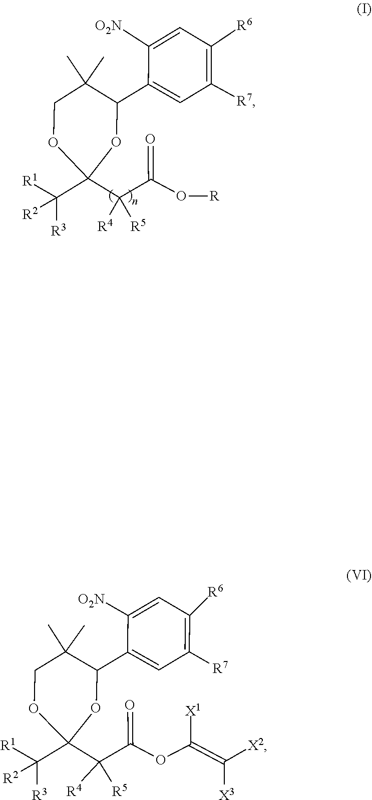

wherein
R is H or D,
R¹, R² and R³ are independently of each other selected from H, D, and a fully or partly deuterated alkyl,
each R⁴ and R⁵ are independently of any other R⁴ or R⁵ selected from H and D,
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-BOc, —O—[CH₂—CH₂—O]ₚ—H, —O—CH₂—CH(OH)—CH₂—OH, —O—CH₂—C(═O)—NH—CH₂—CH₂—NH-Boc, with
   R^{a} being H or a C₁₋₃-alkyl,
   p being an integer between 0 and 6,
X¹, X² and X³ are independently of each other H or D,
n is an integer between 0 and 16.

A fourth aspect of the invention relates to a method for preparing a compound suitable for signal enhanced magnetic resonance imaging. The method comprises the steps of
a) providing a compound of formula (VII),

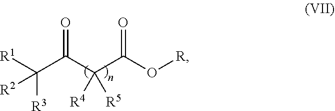

and
   acetylene, wherein 0, 1 or 2 H atoms of acetylene may be replaced by D,
b) reacting the compound of formula (VII) with acetylene in the presence of a metal catalyst yielding a vinyl keto ester of formula (III),

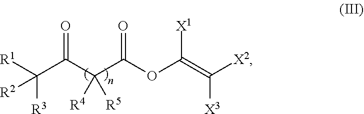

wherein
R¹, R² and R³ are independently of each other selected from H, D, and a fully or partly deuterated alkyl, particularly C₁₋₆-alkyl, more particularly C₁₋₆-alkyl,
each R⁴ and R⁵ are independently of any other R⁴ or R⁵ selected from H and D,
R is H or D,
X¹, X² and X³ are independently of each other H or D, particularly D, and
n is an integer between 0 and 16, particularly between 0 and 6, more particularly between 0 and 3.

### Description

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

The term “alkyl” in the context of the present specification relates to a saturated linear or branched hydrocarbon. For example, a C₁-C₆ alkyl in the context of the present specification relates to a saturated linear or branched hydrocarbon having 1, 2, 3, 4, 5 or 6 carbon atoms. Non-limiting examples for a C₁-C₆ alkyl include methyl, ethyl, propyl, 1-methylethyl (isopropyl), n-butyl, 2-methylpropyl, tert-butyl, n-pentyl, 2-methylbutyl, 3-methylbutyl, 1,1-dimethylpropyl, 1,2-dimethylpropyl, n-hexyl, 3-methyl-2-pentyl, and 4-methyl-2-pentyl. Similarly, the term C₁₋₁₆-alkyl relates to a saturated linear or branched hydrocarbon having 1 to 16 carbon atoms.

The term “hydrocarbon” relates to a compound consisting of C and H atoms. Typically, the number of C atoms is ≤12, particularly ≤8, more particularly ≤6. The compound may be linear, branched or cyclic. The hydrocarbon may comprise one or more double bonds. Non-limiting examples are linear or branched alkyls and benzene.

The term “chlorinated hydrocarbon” relates to a hydrocarbon, wherein at least one H atom is replaced by Cl. Non-limiting examples are chloroform, dichloromethane, dichloroethane, tetrachloroethane, chlorobenzene.

The term “dichloroethane” relates to 1,1-dichloroethane and 1,2-dichloroethane, particularly to 1,2-dichloroethane.

The term “trichloroethane” relates to 1,1,1-trichloroethane and 1,1,2-trichloroethane.

The term “tetrachloroethane” relates to isomers of C₂H₂Cl₄, particularly to 1,1,1,2-tetrachloroethane and 1,1,2,2-tetrachloroethane, more particularly 1,1,2,2-tetrachloroethane.

Unless defined otherwise, all technical and scientific terms used herein have the same meaning as commonly understood by one of ordinary skill in the art.

### DETAILED DESCRIPTION

A first aspect of the invention relates to a vinyl keto ester of formula (III),

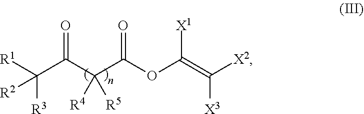

wherein
R¹, R² and R³ are independently of each other selected from H, D, and a fully or partly deuterated alkyl, particularly C₁₋₆-alkyl, more particularly C₁₋₆-alkyl,
each R⁴ and R⁵ are independently of any other R⁴ or R⁵ selected from H and D,
X¹, X² and X³ are independently of each other H or D, wherein at least one of X¹, X² and X³ is D, particularly all of X¹, X² and X³ are D and
n is an integer between 0 and 16, particularly between 0 and 6, more particularly between 0 and 3.

The vinyl keto ester of formula (III) is suitable for the use in signal enhanced magnetic resonance imaging. The vinyl keto ester is a precursor that promotes maximal signal enhancement for metabolites such as pyruvate. To achieve optimal signal enhancement via PHIP not only a ¹³C spins needs to be included into the precursor but ideally at least the vinyl functionality should be deuterated. The compounds according to the first aspect of the invention are characterized by a fully or partly, particularly fully, deuterated vinyl moiety, i.e. at least one of X¹, X² and X³ is D, particularly all of X¹, X² and X³ are D.

For the preparation of a contrast agent for signal enhanced magnetic resonance imaging, a carbon of the vinyl keto ester is hyperpolarized through 1H-polarization transfer via pH₂. The ester obtained may be used as such as contrast agent or may be cleaved by hydrolysis to use the hyperpolarized metabolite such as pyruvate as contrast agent.

In certain embodiments, at least one C atom of the compound of formula (III) is ¹³C.

In certain embodiments, X¹, X² and X³ are D.

In certain embodiments, the compound of formula (III) is partly or fully deuterated.

In certain embodiments, the compound of formula (III) is fully deuterated.

In certain embodiments, the vinyl keto ester of formula (III) is partly or fully deuterated vinyl pyruvate, vinyl ketoisocaproate or vinyl acetoacetate.

Reference is made to the embodiments of the second aspect of the invention, particularly with regard to the definitions of R¹, R², R³, R⁴, R⁵, X¹, X², X³ and n.

The vinyl keto ester as described in the first aspect of the invention may be prepared by a method using a photolabile protecting group or by a method using acetylene. The method using a photolabile protecting group is described in the second aspect of the invention and the method using acetylene is described in the fourth aspect of the invention.

A second aspect of the invention relates to a method for preparing a compound suitable for signal enhanced magnetic resonance imaging. The method comprises the steps of
a) providing a compound of formula (I),

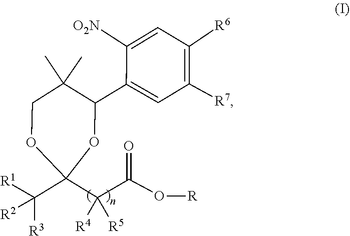

b) vinylation of the compound of formula (I) by using vinyl acetate of formula (II),

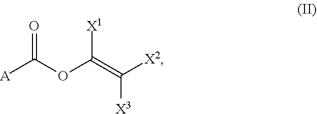

c) applying UV light yielding a vinyl keto ester of formula (III),

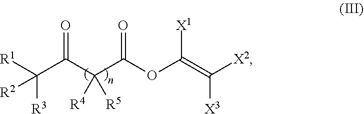

wherein
R is H or D, particularly H,
R¹, R² and R³ are independently of each other selected from H, D, and a fully or partly deuterated alkyl, particularly C₁₋₆-alkyl, more particularly C₁₋₆-alkyl, even more particularly C₁₋₃-alkyl,
each R⁴ and R⁵ are independently of any other R⁴ or R⁵ selected from H and D, particularly D,
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-BOc, —O—[CH₂—CH₂—O]ₚ—H, —O—CH₂—CH(OH)—CH₂—OH, —O—CH₂—C(═O)—NH—CH₂—CH₂—NH-Boc, with
   R^{a} being H or a C₁₋₃-alkyl,
   p being an integer between 0 and 6,
X¹, X² and X³ are independently of each other H or D, particularly D,
A is —CH₃, —CH₂D, —CHD₂ or -CD₃, particularly —CD₃, and
n is an integer between 0 and 16, particularly between 0 and 6, more particularly between 0 and 3.

The method described in the second aspect of the invention aims to provide a vinyl keto ester that is suitable for the use in signal enhanced magnetic resonance imaging. The vinyl keto ester is a precursor that promotes maximal signal enhancement for metabolites such as pyruvate. To achieve optimal signal enhancement via PHIP not only a ¹³C spins needs to be included into the precursor but ideally at least the vinyl functionality should be deuterated.

Known attempts to produce suitable vinyl keto esters suffer from low yields (ca. 10%) and loss of deuterated protons.

The method of the second aspect of the invention makes use of a photo-cleavable protecting group that allows vinylation under mild conditions without loss of deuterium. Very good yields are achieved (>80%).

For the preparation of a contrast agent for signal enhanced magnetic resonance imaging, a carbon of the vinyl keto ester is hyperpolarized through 1H-polarization transfer via pH₂. The ester obtained may be used as such as contrast agent or may be cleaved by hydrolysis to use the hyperpolarized metabolite such as pyruvate as contrast agent.

In certain embodiments, R¹, R² and R³ are independently of each other selected from H, D, and a fully or partly deuterated C₁₋₃-alkyl.

In certain embodiments, R¹ and R² are H or D, and R³ is selected from H, D and a fully or partly deuterated alkyl, particularly C₁₋₆-alkyl, more particularly C₁₋₆-alkyl, even more particularly C₁₋₃-alkyl.

In certain embodiments, R¹ and R² are H or D, and R³ is selected from H, D and a fully or partly deuterated C₃-alkyl, particularly selected from H, D and isopropyl.

To reduce the risk of loss of deuterated protons, the keto ester moiety may be fully deuterated.

R¹, R² and R³ are independently of each other selected from D, and a fully deuterated alkyl, particularly a fully deuterated C₁₋₆-alkyl, more particularly a fully deuterated C₁₋₆-alkyl.

In certain embodiments, R¹, R² and R³ are independently of each other selected from D, and a fully deuterated C₁₋₃-alkyl.

In certain embodiments, R¹ and R² are D, and R³ is selected from D and a fully deuterated alkyl, particularly C₁₋₆-alkyl, more particularly C₁₋₆-alkyl, even more particularly C₁₋₃-alkyl.

In certain embodiments, R¹ and R² are D, and R³ is selected from D and fully deuterated C₃-alkyl, particularly selected from D and isopropyl.

In certain embodiments, R is D.

In certain embodiments, R⁴ and R⁵ are D.

In certain embodiments, n is 0 or 1.

In certain embodiments, X¹, X² and X³ are D.

In certain embodiments, A is —CD₃.

In certain embodiments, the compound of formula (I) is prepared by protecting a compound of formula (IV),

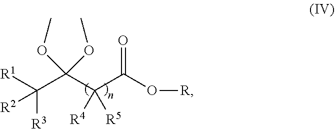

using a protecting group of formula (V),

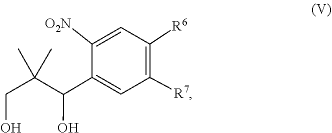

wherein R, R¹, R², R³, R⁴, R⁵, R⁶, R⁷ and n are defined as described above.

To achieve maximal signal enhancement, the compound of formula (IV) should be ¹³C-enriched.

In certain embodiments, one or more C atoms of the compound of formula (IV) are ¹³C atoms.

In certain embodiments, R⁶ is H or —OCH₃, and R⁷ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with R^{a} being H or a C₁₋₃-alkyl, and p being an integer between 0 and 6.

Suitable protecting groups are compounds of formula (V) with R⁶ and R⁷ being H as well as the following compounds:

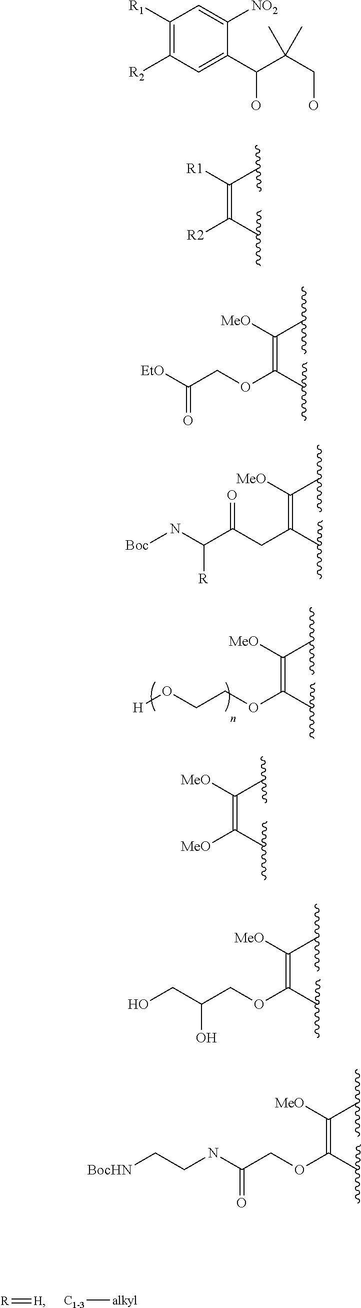

In certain embodiments, the preparation of a compound of formula (I) is performed in toluene.

To increase the yield of the compound of formula (III), the vinylation in step (b) may be performed repeatedly. Both, unreacted starting material (compound of formula (I)) and unreacted deuterated vinyl acetate (compound of formula (II)) can be recovered from the reaction and recycled.

In certain embodiments, the vinylation in step (b) is performed repeatedly.

The vinylation is performed in the presence of a catalyst for transfer esterification such as a Pd(0) and/or Pd(2+) catalyst.

In certain embodiments, the vinylation in step (b) is performed using Pd(OAc)₂.

The protecting group is cleaved by applying UV light in step (c).

In certain embodiments, the UV light in step (c) has a wave length between 200 nm and 500 nm, particularly 365 nm.

For the preparation of a contrast agent for signal enhanced magnetic resonance imaging, a carbon of the vinyl keto ester is hyperpolarized through ¹H-¹³C-polarization transfer via pH₂ by standard methods The ester obtained may be used as such as contrast agent or may be cleaved by hydrolysis to use the hyperpolarized metabolite such as pyruvate as contrast agent.

In certain embodiments, the vinyl keto ester of formula (III) is hyperpolarized.

In certain embodiments, the vinyl keto ester of formula (III) is hyperpolarized and hydrolysed after step (c).

In certain embodiments, at least one C atom of the compound of formula (III) is ¹³C.

In certain embodiments, steps (a), (b) and (c) are performed at a temperature between 15° C. and 35° C., particularly between 20° C. and 25° C.

A third aspect of the invention relates to a compound of formula (I) or (VI),

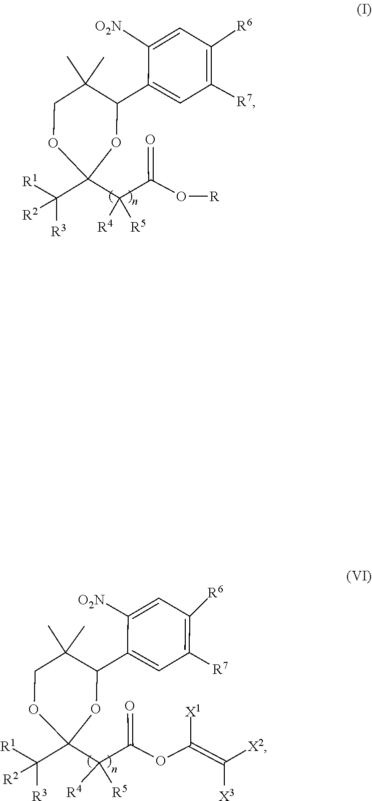

wherein
R is H or D, particularly H,
R¹, R² and R³ are independently of each other selected from H, D, and a fully or partly deuterated alkyl, particularly C₁₋₁₆-alkyl, more particularly C₁₋₆-alkyl,
each R⁴ and R⁵ are independently of any other R⁴ or R⁵ selected from H and D,
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
   R^{a} being H or a C₁₋₃-alkyl,
   p being an integer between 0 and 6,
X¹, X² and X³ are independently of each other H or D,
n is an integer between 0 and 16, particularly between 0 and 6, more particularly between 0 and 3.

In certain embodiments, at least one C atom of the moiety

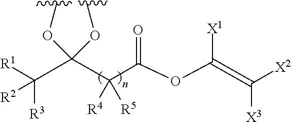

### is ¹³C.

In certain embodiments, at least one of X¹, X² and X³ is D.

In certain embodiments, X¹, X² and X³ are D.

Reference is made to the embodiments of the first and second aspect of the invention, particularly with regard to the definitions of R, R¹, R², R³, R⁴, R⁵, R⁶, R⁷, X¹, X², X³ and n.

A fourth aspect of the invention relates to a method for preparing a compound suitable for signal enhanced magnetic resonance imaging. The method comprises the steps of
a) providing a compound of formula (VII),

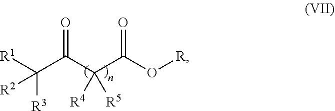

and
   acetylene, wherein 0, 1 or 2 H atoms of acetylene may be replaced by D,
b) reacting the compound of formula (VII) with acetylene in the presence of a metal catalyst yielding a vinyl keto ester of formula (III),

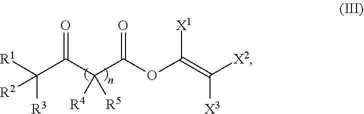

wherein
R¹, R² and R³ are independently of each other selected from H, D, and a fully or partly deuterated alkyl, particularly C₁₋₆-alkyl, more particularly C₁₋₆-alkyl,
each R⁴ and R⁵ are independently of any other R⁴ or R⁵ selected from H and D,
R is H or D,
X¹, X² and X³ are independently of each other H or D, particularly D, and
n is an integer between 0 and 16, particularly between 0 and 6, more particularly between 0 and 3.

The transferesterification approach described in the second aspect of the invention requires multiple steps and a large supply of deuterated ester from which the vinyl group is transferred to the desired product. Hence, this approach may not be easily applicable for a large-scale production.

The method according to the fourth aspect of the invention allows to synthesize vinyl esters of keto acids by using acetylene and a suitable solvent and a suitable catalyst in a one-step reaction. The method is applicable for non-enriched compounds as well as partly or fully deuterated keto esters as well as esters enriched with one or multiple ¹³C or different oxygen isotopes. If the keto ester is deuterated and comprises at least one ¹³C atom, it may be used for signal enhanced magnetic resonance imaging as described in the second aspect of the invention.

To obtain a vinyl keto ester that comprises a fully deuterated vinyl moiety, a deuterated compound of formula (VII) and deuterated acetylene are used.

The deuterated compound of formula (VII), e.g. deuterated pyruvate, can be obtained by using D₂O to exchange protons with deuterium. To avoid the loss of deuterium, the solvent and additives that are used for the reaction with acetylene should be deuterated. Also deuterated acetylene may be used.

In certain embodiments, R is D. In certain embodiments, R¹, R² and R³ are independently selected from D, and a fully or partly, particularly fully, deuterated alkyl, and R⁴ R⁵ and R are D.

In certain embodiments, acetylene is fully deuterated, i.e. 2 H atoms are replaced by D.

In certain embodiments, R is D and acetylene is fully deuterated.

In certain embodiments, R¹, R² and R³ are independently selected from D, and a fully or partly, particularly fully, deuterated alkyl, and R⁴ R⁵ and R are D and acetylene is fully deuterated.

Although the reaction of acetylene gas with acids has been explored in the past, so far it has not been used to obtain vinylated keto esters and most importantly not vinyl pyruvate. In WO 2010/129030 A2 only formation of vinyl benzoate (VB), vinyl 2-ethyl hexanoate, and vinyl esters of various other neo carboxylic acids using homogeneous catalysts of platinum group metals was shown. The reaction was done in neat carboxylic acid or in several solvents that are acetonitrile, benzonitrile, butyl benzoate, mineral oil, diethylene glycol dibutylether and toluene.

The inventors believe that the following reasons were hindering the synthesis of vinyl keto esters such as vinyl pyruvate. The pyruvic acid itself is unstable and decomposes at high temperature. Moreover, resulting vinyl pyruvate is even more unstable starting to decompose above 60° C. which is accelerated by the presence of metal catalysts. This results in the production of CO₂ that dilutes acetylene and slows further the desired reaction path while the substrate continues to decompose with the same rate. Most of the developed syntheses require high temperatures for sufficiently rapid production of vinyl esters. The solvent should be chosen carefully because of the reactivity of pyruvic acid with ketones. The same applies for neat pyruvic acid without any solvent.

The reaction according to the fourth aspect of the invention may be performed using the neat compound of formula (VII) without any solvent or using the compound of formula (VII) dissolved in a suitable solvent.

In certain embodiments, the compound of formula (VII) is provided as neat compound or dissolved in a solvent.

In certain embodiments, the compound of formula (VII) is used in step (b) as neat compound. For the method according to the fourth aspect of the invention, the solvent plays a significant role. Most of the proposed solvents for reactions using acetylene (e.g. solvents disclosed in WO 2010/129030 A) were found to slow the reaction. The best solvents for the vinyl pyruvate production in terms of rate and low side reactions are chlorinated solvents, particularly 1,1,2,2-tetrachloroethane. Previously these solvents were not considered, probably for the reason of the low acetylene solubility.

In certain embodiments, the compound of formula (VII) is dissolved in a suitable solvent.

If deuterated vinyl keto esters are prepared, the solvent may be deuterated to prevent loss of deuterium. In certain embodiments, the solvent is deuterated.

In certain embodiments, the compound of formula (VII) is dissolved in a solvent selected from a chlorinated hydrocarbon, a chlorinated ether, a chlorinated acetophenone, acetonitrile, acetic acid, an ether, an ester, toluene, acetone, ethanol or a mix thereof, wherein the solvent may optionally be fully or partly deuterated.

If a mix of solvents is used, a chlorinated hydrocarbon, particularly chloroform, is mixed with toluene or ethanol.

In certain embodiments, the compound of formula (VII) is dissolved in a solvent selected from a chlorinated hydrocarbon, a chlorinated ether, a chlorinated acetophenone, acetonitrile, acetic acid, an ether, an ester, toluene, acetone, wherein the solvent may optionally be fully or partly deuterated.

In certain embodiments, the compound of formula (VII) is dissolved in a solvent selected particularly from chlorinated hydrocarbon, a chlorinated ether, a chlorinated acetophenone or a mix thereof,

In certain embodiments, the compound of formula (VII) is dissolved in a solvent selected from a chlorinated hydrocarbon, a chlorinated ether, a chlorinated acetophenone, wherein the solvent may optionally be fully or partly deuterated.

In certain embodiments, the solvent is selected from chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, acetonitrile, acetic acid, diethyl ether, dibenzyl ether, ethyl acetate, butyl benzoate, toluene, acetone, ethanol or a mix thereof, wherein the solvent may optionally be fully or partly deuterated.

In certain embodiments, the solvent is selected from chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, acetonitrile, acetic acid, diethyl ether, dibenzyl ether, ethyl acetate, butyl benzoate, toluene, acetone, wherein the solvent may optionally be fully or partly deuterated.

In certain embodiments, the solvent is selected from chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, diethyl ether, wherein the solvent may optionally be fully or partly deuterated.

In certain embodiments, the solvent is selected from chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, wherein the solvent may optionally be fully or partly deuterated.

In certain embodiments, the solvent is selected from chloroform (CHCl₃), dichloromethane (CH₂Cl₂), dichloroethane, tetrachloroethane, 4-chloroacetophenone, 4-chloroanisole and chlorobenzene.

In certain embodiments, the solvent is selected from chloroform (CHCl₃), dichloromethane (CH₂Cl₂), dichloroethane, tetrachloroethane, 4-chloroanisole and toluene.

In certain embodiments, the solvent is selected from chloroform (CHCl₃), dichloromethane (CH₂Cl₂), dichloroethane, tetrachloroethane, 4-chloroanisole.

In certain embodiments, the solvent is selected from chloroform (CHCl₃), dichloromethane (CH₂Cl₂), tetrachloroethane, and chlorobenzene.

In certain embodiments, the solvent is chloroform or tetrachloromethane.

In certain embodiments, the solvent is tetrachloromethane, particularly 1,1,2,2-tetrachloroethane.

As catalyst any complex of the platinum group metal or rhenium can be used. However, best results with highest yield and small decomposition rate were achieved using bis(1,5-cyclooctadiene)diiridium(I) dichloride ([Ir(1,5-cod)Cl]₂) or (1,5-Cyclooctadiene)(methoxy)iridium(I) dimer.

In certain embodiments, the metal catalyst is selected from an iridium catalyst, a rhodium catalyst, a ruthenium catalyst, a palladium catalyst, an osmium catalyst, a platinum catalyst and a rhenium catalyst.

In certain embodiments, the metal catalyst is selected from an iridium(I) catalyst and a rhodium(I) catalyst.

In certain embodiments, the metal catalyst is selected from 1,5-cyclooctadien-iridium(I)-chloride-dimer, (1,5-cyclooctadiene) (hexafluoroacetylacetonato) iridium(I), acetylacetonato(1,5-cyclooctadiene)iridium(1), (1,5-Cyclooctadiene)(methoxy)iridium(I) dimer, dichloro(p-cymene)ruthenium(II) dimer, tris(triphenylphosphine)ruthenium(II) dichloride, [1,4-bis(diphenylphosphino)butane](1,5-cyclooctadiene)rhodium(1) tetrafluoroborate.

In certain embodiments, the metal catalyst is selected from 1,5-cyclooctadien-iridium(I)-chloride-dimer, (1,5-cyclooctadiene) (hexafluoroacetylacetonato) iridium(I), acetylacetonato(1,5-cyclooctadiene)iridium(1), (1,5-Cyclooctadiene)(methoxy)iridium(I) dimer, [1,4-bis(diphenylphosphino)butane](1,5-cyclooctadiene)rhodium(1) tetrafluoroborate.

Particularly for reactions with the neat compound of formula (VII) without any solvents, an iridium catalyst, particularly (1,5-Cyclooctadiene)(methoxy)iridium(1) dimer, is used.

In certain embodiments, step (b) is performed without any solvent and the catalyst is an iridium catalyst, particularly (1,5-Cyclooctadiene)(methoxy)iridium(I) dimer.

In certain embodiments, step (b) is performed without any solvent and the catalyst is an iridium catalyst, particularly (1,5-Cyclooctadiene)(methoxy)iridium(I) dimer and/or step (b) is performed using the compound of formula (VII) dissolved in a solvent as described above, particularly a solvent selected from chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, acetonitrile, acetic acid, diethyl ether, dibenzyl ether, ethyl acetate, butyl benzoate, toluene, acetone, ethanol or a mix thereof, more particularly chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, wherein the solvent may optionally be fully or partly deuterated and the catalyst is selected from an iridium catalyst, a rhodium catalyst, a ruthenium catalyst, a palladium catalyst, an osmium catalyst, a platinum catalyst and a rhenium catalyst, particularly an iridium(I) catalyst and a rhodium(I) catalyst, more particularly the metal catalyst is selected from 1,5-cyclooctadien-iridium(I)-chloride-dimer, (1,5-cyclooctadiene) (hexafluoroacetylacetonato) iridium(I), acetylacetonato(1,5-cyclooctadiene)iridium(I), (1,5-Cyclooctadiene)(methoxy)iridium(I) dimer, dichloro(p-cymene)ruthenium(II) dimer, tris(triphenylphosphine)ruthenium(II) dichloride, [1,4-bis(diphenylphosphino)butane](1,5-cyclooctadiene)rhodium(I) tetrafluoroborate.

In certain embodiments, step (b) is performed using the compound of formula (VII) dissolved in a solvent as described above, particularly a solvent selected from chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, acetonitrile, acetic acid, diethyl ether, dibenzyl ether, ethyl acetate, butyl benzoate, toluene, acetone, ethanol or a mix thereof, more particularly chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, wherein the solvent may optionally be fully or partly deuterated and the catalyst is selected from an iridium catalyst, a rhodium catalyst, a ruthenium catalyst, a palladium catalyst, an osmium catalyst, a platinum catalyst and a rhenium catalyst, particularly an iridium(I) catalyst and a rhodium(I) catalyst, more particularly the metal catalyst is selected from 1,5-cyclooctadien-iridium(I)-chloride-dimer, (1,5-cyclooctadiene) (hexafluoroacetylacetonato) iridium(I), acetylacetonato(1,5-cyclooctadiene)iridium(I), (1,5-Cyclooctadiene)(methoxy)iridium(I) dimer, dichloro(p-cymene)ruthenium(II) dimer, tris(triphenylphosphine)ruthenium(II) dichloride, [1,4-bis(diphenylphosphino)butane](1,5-cyclooctadiene)rhodium(I) tetrafluoroborate.

Keto esters and especially vinyl pyruvate can be synthesized via the pathway described in the fourth aspect of the invention only at mild conditions. The temperature is crucial for the whole process, particularly when preparing vinyl pyruvate. Pyruvic acid itself is unstable and decomposes at high temperature. Moreover, resulting vinyl pyruvate is even more unstable starting to decompose above 60° C. which is accelerated by the presence of metal catalysts. This results in the production of CO₂ that dilutes acetylene and slows further the desired reaction path while the substrate continues to decompose with the same rate. Most of known syntheses require high temperatures for sufficiently rapid production of vinyl esters.

The method according to the fourth aspect of the invention should be performed at a temperature below 160° C.

Above 60° C. and below 160° C., the decomposition of substrate and product is fast enough that only 50% yield is possible. However, such conditions may still be applied if a fast production is needed.

The reaction may be performed in a reactive distillation column where the product is removed from the high temperature zone thus preventing its decay and shifting the whole reaction towards the product to obtain a high yield.

In certain embodiments, step (b) is performed at a temperature ≤160° C.

To avoid the decomposition of substrate and product, the reaction may be performed at a temperature below 60° C. Under such conditions, the process takes several days to complete but high yields can be achieved.

In certain embodiments, step (b) is performed at a temperature ≤60° C.

In certain embodiments, step (b) is performed without any solvent and the catalyst is an iridium catalyst, particularly (1,5-Cyclooctadiene)(methoxy)iridium(I) dimer and step (b) is performed at a temperature ≤60° C.

In certain embodiments, step (b) is performed without any solvent and the catalyst is an iridium catalyst, particularly (1,5-Cyclooctadiene)(methoxy)iridium(I) dimer and/or step (b) is performed using the compound of formula (VII) dissolved in a solvent as described above, particularly a solvent selected from chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, acetonitrile, acetic acid, diethyl ether, dibenzyl ether, ethyl acetate, butyl benzoate, toluene, acetone, ethanol or a mix thereof, more particularly chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, wherein the solvent may optionally be fully or partly deuterated and the catalyst is selected from an iridium catalyst, a rhodium catalyst, a ruthenium catalyst, a palladium catalyst, an osmium catalyst, a platinum catalyst and a rhenium catalyst, particularly an iridium(I) catalyst and a rhodium(I) catalyst, more particularly the metal catalyst is selected from 1,5-cyclooctadien-iridium(I)-chloride-dimer, (1,5-cyclooctadiene) (hexafluoroacetylacetonato) iridium(I), acetylacetonato(1,5-cyclooctadiene)iridium(I), (1,5-Cyclooctadiene)(methoxy)iridium(1) dimer, dichloro(p-cymene)ruthenium(II) dimer, tris(triphenylphosphine)ruthenium(II) dichloride, [1,4-bis(diphenylphosphino)butane](1,5-cyclooctadiene)rhodium(1) tetrafluoroborate, and step (b) is performed at a temperature ≤60° C.

In certain embodiments, step (b) is performed using the compound of formula (VII) dissolved in a solvent as described above, particularly a solvent selected from chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, acetonitrile, acetic acid, diethyl ether, dibenzyl ether, ethyl acetate, butyl benzoate, toluene, acetone, ethanol or a mix thereof, more particularly chloroform (CHCl₃), dichloromethane (CH₂Cl₂), chloromethane (CH₃Cl), dichloroethane, trichloroethane, tetrachloroethane, 4′-chloroacetophenone, 4-chloroanisole and chlorobenzene, wherein the solvent may optionally be fully or partly deuterated and the catalyst is selected from an iridium catalyst, a rhodium catalyst, a ruthenium catalyst, a palladium catalyst, an osmium catalyst, a platinum catalyst and a rhenium catalyst, particularly an iridium(I) catalyst and a rhodium(I) catalyst, more particularly the metal catalyst is selected from 1,5-cyclooctadien-iridium(I)-chloride-dimer, (1,5-cyclooctadiene) (hexafluoroacetylacetonato) iridium(I), acetylacetonato(1,5-cyclooctadiene)iridium(I), (1,5-Cyclooctadiene)(methoxy)iridium(1) dimer, dichloro(p-cymene)ruthenium(II) dimer, tris(triphenylphosphine)ruthenium(II) dichloride, [1,4-bis(diphenylphosphino)butane](1,5-cyclooctadiene)rhodium(1) tetrafluoroborate, and step (b) is performed at a temperature ≤60° C.

To avoid polymerization reactions, a polymerization inhibitor may be added to the reaction mixture. Any known polymerization inhibitor may be used. Non-limiting examples are hydroquinone, quinine and catechol.

In certain embodiments, step (b) is performed in the presence of a polymerization inhibitor.

In certain embodiments, step (b) is performed in the presence of a polymerization inhibitor selected from hydroquinone, quinine and catechol.

The reactivity of the catalyst may be tuned by using additives.

In certain embodiments, step (b) is performed in the presence of an additive selected from Na₂CO₃, sodium pyruvate (NaPyr) and benzoic acid.

However, the process works best with the neat catalyst.

In certain embodiments, step (b) is performed without an additive that modulates the reactivity of the catalyst.

This synthesis route offers to produce vinyl ketoesters such as vinyl pyruvate with hydrogen and natural abundant isotopes or isotopically enriched derivatives with any combination of ¹H, ²H and ¹³C and in some rare case different oxygen isotopes.

The substrates can be partially or completely deuterated or labeled with ¹³C to produce labeled vinyl pyruvate.

For the preparation of a contrast agent for signal enhanced magnetic resonance imaging, a carbon of the vinyl keto ester is hyperpolarized through 1H-polarization transfer via pH₂. The ester obtained may be used as such as contrast agent or may be cleaved by hydrolysis to use the hyperpolarized metabolite such as pyruvate as contrast agent.

In certain embodiments, at least one C atom of the compound of formula (III) is ¹³C.

In certain embodiments, X¹, X² and X³ are D.

In certain embodiments, the compound of formula (III) is partly or fully deuterated.

In certain embodiments, the compound of formula (III) is fully deuterated.

Reference is made to the embodiments of the first and second aspect of the invention, particularly with regard to the definitions of R, R¹, R², R³, R⁴, R⁵, X¹, X², X³ and n.

In certain embodiments of any aspect of the present invention, one or more protons are replaced by deuterium. The molecules described herein may be fully deuterated.

The invention is further illustrated by the following examples, from which further embodiments and advantages can be drawn. These examples are meant to illustrate the invention but not to limit its scope.

### EXAMPLES

### Example 1: Synthesis of Vinyl Keto Esters Using a Photocleavable Protecting Group

Deuterated vinyl pyruvate (8) was synthesized using a photocleavable protecting group strategy as shown in Scheme 1 and described below.

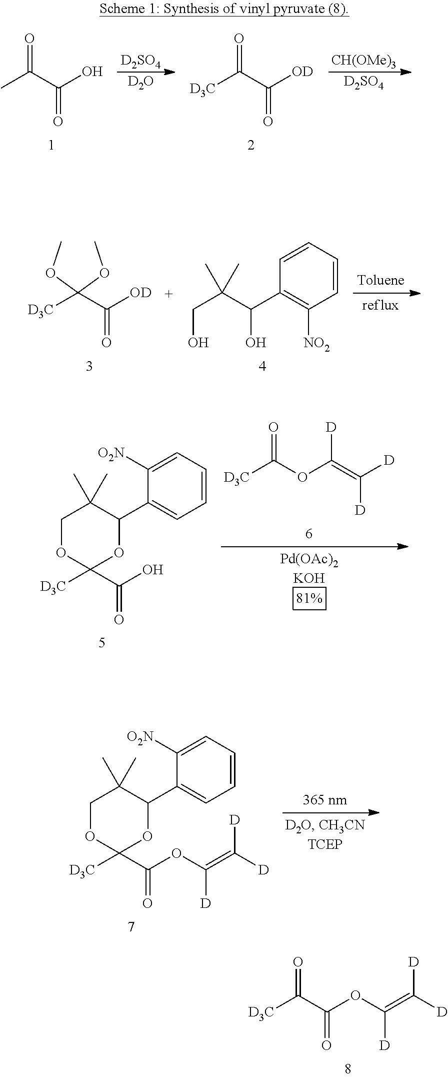

### Synthesis of Compound 2: Deuteration of Pyruvic Acid

This step is optional, deuteration of the pyruvate can however lead to larger signal enhancements.

To a cold solution of pyruvic acid (0.5 g, 5.6 mmol) in D₂O (6 mL), D₂SO₄ (0.135 mL, 2.45 mmol, 98% D₂SO₄ in D₂O) was added dropwise. The resulting solution was heated to reflux for 2 h. The reaction mixture was cooled down to rt and NaCl was added until the solution was saturated. The aqueous phase was extracted with diethyl ether (20×15 mL). The combined organic layers were dried over Na₂SO₄, filtered, and concentrated cautiously in vacuo at 30° C. to get 2 as an oil with quantitative yields. ²H-NMR analysis of 2 showed 75% CD₃ and 25% CD₂H groups in pyruvic acid. In order to get full deuteration, the above reaction was performed one more time.

Alternatively, deuterated pyruvate may be obtained from commercial sources in ¹³C enriched or non-enriched form.

### Synthesis of Compound 3

A mixture of 2 (0.79 mL, 11.6 mmol) and trimethyl orthoformate (4.5 mL, 40.7 mmol) was stirred at 10° C. To this cold solution, sulfuric acid (0.057 mL, 1.06 mmol) was added dropwise. The resulting solution was stirred at 5-10° C. for 70 min and then quenched by the addition of brine (15 mL). The reaction mixture was extracted with dichloromethane (3×15 mL). The combined organic layers were dried over Na₂SO₄, filtered, and concentrated in vacuo to get 3 as colorless oil.

### Synthesis of Compound 5

A solution of 3 (1.16 g, 8.63 mmol) and 4 (2.33 g, 10.4 mmol) in toluene (30 mL) was heated to reflux. After 2 h the solution was cooled down to rt. The organic solvent was removed in vacuo to obtain the crude product, which was washed with water (20 mL). The aqueous phase was extracted with CH₂Cl₂ (3×15 mL). The combined organic layers were dried over Na₂SO₄, filtered, and concentrated in vacuo. The crude material was purified by flash column chromatography (silica) using a gradient solvent system (MeOH:CH₂Cl₂; 0:100 to 5:95) to yield 5 as an off-white solid.

The photoprotection group derived from 4 needs to be introduced to obtain high yields in the following reaction for compound 6.

### Synthesis of Compound 7: Vinylation Reaction

Obtaining high yields for the vinylation reactions with deuterated starting material is a key point to obtain deuterated vinyl pyruvate after all steps.

A mixture of 5 (1 g, 3.34 mmol), Pd(OAc)₂ (0.008 g, 0.0334 mmol), KOH (0.018 g, 0.334 mmol) and vinyl acetate-d₆ (6, 5 mL) was stirred for 24 h at rt under N₂. The unconverted vinyl acetate-d₆ (6) was recovered by short path distillation. The reaction mixture was diluted in dichloromethane and washed with D₂O (15 mL). The organic layer was dried over Na₂SO₄, filtered, and concentrated in vacuo. The crude material was purified by flash column chromatography (silica) using a gradient solvent system (EtOAc:petroleum ether; 0:100 to 5:95) to yield 7 as colourless liquid.

Both, unreacted starting material and unreacted deuterated vinyl acetate can be recovered from the reaction and recycled. Starting material (5) was recovered (0.43 g) by washing the column with 10% MeOH in CH₂Cl₂. Recovered starting material (5) and recovered vinyl acetate (6) was subjected to the vinylation reaction one more time to obtain 7 (0.89 g, 81% as combined yield) as colourless liquid.

### Synthesis of Compound 8

Removal of the photoprotection group as described in the following leads to the desired vinyl ester.

To a clear solution of 7 (0.42 g, 1.56 mmol) in CH₃CN (20 mL), Tris(2-carboxyethyl)phosphine hydrochloride (TCEP, 2.5 mL, 0.1 M in H₂O) was added in D₂O (3.7 mL). The solution was irradiated with UV light (365 nm, LED bulb) from a monochromatic light source for 2 h. The reaction mixture was diluted with CH₂Cl₂ (25 mL) and washed with D₂O (30 mL). The organic layer was dried over Na₂SO₄, filtered, and concentrated cautiously in vacuo at 30° C. The crude material was purified by distillation on Kugelrohr distillation under reduced pressure (250 mbar) at 75° C. heater temperature—to get 8 as a colorless oil.

### Hyperpolarization

Compound 8 was hyperpolarized and cleaved as shown in Scheme 2 to obtain an NMR contrast agent.

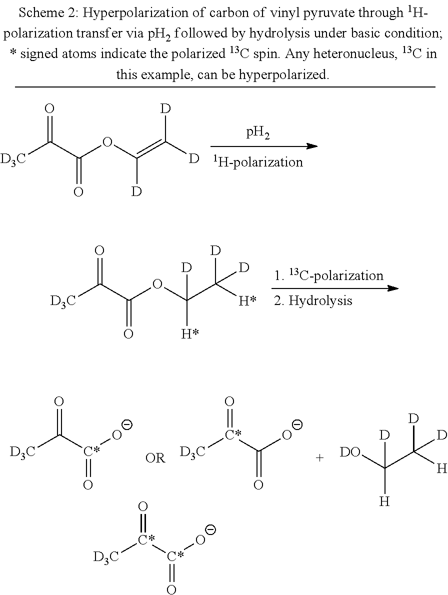

### Example 2: Synthesis of Vinyl Keto Esters Using Acetylene

Vinyl pyruvate was synthesized as shown in Scheme 3 and described below. The general scheme is also applicable for the production of vinyl pyruvate labeled with deuterium and/or ¹³C. For the synthesis of vinyl pyruvate having a deuterated vinyl moiety, the synthesis is started with pyruvate having a —COOD moiety instead of the —COOH moiety as shown in Scheme 3. The deuterated pyruvate can be obtained by using D₂O to exchange the proton with deuterium. Similarly, the —CH₃ moiety can be transformed to —CD₃. To avoid the loss of deuterium, the solvent and additives that are used for the reaction with acetylene can be deuterated. Also deuterated acetylene may be used.

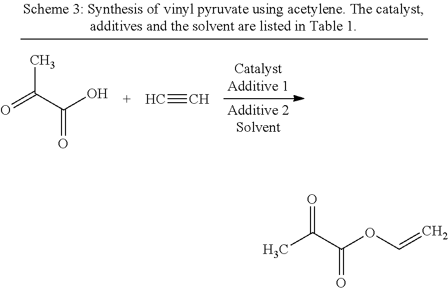

The substrates can be partially or completely deuterated or labeled with ¹³C to produce labeled vinyl pyruvate.

As catalyst any complex of the platinum group metal or rhenium can be used. However, best results with highest yield and small decomposition rate were achieved using bis(1,5-cyclooctadiene)diiridium(I) dichloride ([Ir(1,5-cod)Cl]₂) and (1,5-Cyclooctadiene)(methoxy)iridium(I) dimer.

Additive 1 (see Table 1) is used to tune the catalyst reactivity. Several additives were tried but with the neat catalyst the process works best. If the reaction is performed to obtain deuterated vinyl pyruvate, the additive 1 can be deuterated to avoid the loss of deuterium. For instance, deuterated ethanol may be used.

Additive 2 is a polymerization inhibiter. Hydroquinone, quinine and catechol were successfully used, however, any known polymerization inhibiter can also be used. If the reaction is performed to obtain deuterated vinyl pyruvate, the additive 2 can be deuterated to avoid the loss of deuterium.

Several solvents and catalysts were tested with regard to their impact on the reaction rate (see Table 1). The reaction was monitored by means of NMR measurements to quantify the ratio of educts and products at different times.

The solvent plays a significant role in the reaction. Most of the proposed solvents for reactions using acetylene (e.g. solvents disclosed in WO 2010/129030 A) were found to slow the reaction. The best solvents for the vinyl pyruvate production in terms of rate and low side reactions are chlorinated solvents, particularly 1,1,2,2-tetrachloroethane. Previously these solvents were not considered, probable for the reason of the low acetylene solubility.

Alternatively, neat pyruvate may be used. The reaction without any solvent may be performed when using (1,5-cyclooctadiene)(methoxy)iridium(I) dimer as catalyst. The reaction is slow compared e.g. to the reaction performed in a chlorinated solvent such as chloroform or tetrachloroethane. However, production costs may be reduced by choosing reaction conditions without any solvent.

The temperature is crucial for the whole process. Above 60° C. the decomposition of substrate and product is fast enough that only 50% yield is possible. However, below this temperature the process takes several days to complete.

This synthesis route offers to produce vinyl ketoesters/vinyl pyruvate with hydrogen and natural abundant isotopes or isotopically enriched derivatives with any combination of ¹H, ²H and ¹³C and in some rare case different oxygen isotopes.

TABLE 1
Relative reaction rates of pyruvic acid
with acetylene to produce vinyl pyruvate.

| Catalyst | Solvent | Additive 1 | Reaction rate |
|---|---|---|---|
| [Ir(cod)Cl]₂ | —, neat pyruvic acid | — | Phase separation |
| | Chloroform | — | Fast |
| | | Na₂CO₃ | Fast |
| | | NaPyr | Fast |
| | Chloroform + Toluene | — | Very slow |
| | Chloroform + 1% | — | Very slow and |
| | Ethanol | | side reaction |
| | Toluene | — | Very slow |
| | Dichloromethane | — | Fast |
| | Dichloromethane | Na₂CO₃ | Fast |
| | Ethyl acetate | — | Slow |
| | Dichloroethane | — | Fast |
| | Chlorobenzene | — | Fast |
| | Tetrachloroethane | — | Fast |
| | Acetone | — | Fast, proton/ |
| | | | deuterium |
| | | | exchange with |
| | | | pyruvate |
| | Acetic acid | — | Slow |
| | 1,2-dichlorobenzene | — | Phase separation |
| | Butyl benzoate | — | Very slow |
| | Acetonitrile | — | Very slow |
| | Diethyl ether | — | Fast |
| | Butyl Phenyl Ether | — | Phase separation |
| | 4′-Chloroacetophenone | — | Fast |
| | 4-chloroanisole | — | Fast |
| [Ir(cod)OMe]₂ | —, neat | — | Slow |
| | Chloroform | — | Very fast |
| | Tetrachloroethane | — | Very fast |
| | Acetone | — | Very fast, |
| | | | proton/deuterium |
| | | | exchange with |
| | | | pyruvate |
| | Acetonitrile | — | Very slow |
| | 4′-Chloroacetophenone | — | Fast |
| | 4-chloroanisole | — | Fast |
| | Diphenyl ether | — | No |
| | Dibenzyl ether | — | Slow |
| IrAcAc | Chloroform | — | Very slow |
| IrAcAcF6 | Chloroform | — | Very slow |
| Ru cumene | Chloroform | — | Very slow |
| | Chloroform | Benzoic acid | Slow, and |
| | | | side product |
| | Ethylacetate | — | No |
| | Acetonitrile | — | No, Side product |
| Ru phosphine | Chloroform | — | Very slow |
| Rh | Chloroform | — | Fast |
| | Ethyl acetate | — | No |
| | Acetonitrile | — | No |
| | Chloroform | Benzoic acid | Fast |

[Ir(cod)Cl]₂ = 1,5-Cyclooctadien-iridium(I)-chlorid-dimer; IrAcAcF6 = (1,5-Cyclooctadiene)(hexafluoroacetylacetonato)iridium(I); [Ir(cod)OMe]₂ = (1,5-Cyclooctadiene)(methoxy)iridium(I) dimer; IrAcAc = Acetylacetonato(1,5-cyclooctadiene)iridium(I); Ru cumene = Dichloro(p-cymene)ruthenium(II) dimer; Ru phosphine = Tris(triphenylphosphine)ruthenium(II) dichloride; Rh = [1,4-Bis(diphenylphosphino)butane](1,5-cyclooctadiene)rhodium(I) tetrafluoroborate

### Synthesis of Vinyl Pyruvate in Tetrachloroethane Using the catalyst [Ir(COD)Cl]₂

1 ml of pyruvic acid is dissolved in 1 ml tetrachloroethane and 10 mg of the catalyst [Ir(COD)Cl]₂ is added. The solution is subsequently degassed and pressurized with 1.8 bar of acetylene gas. The reaction is carried out under constant acetylene pressure at 37° C. After 3 days pyruvic acid is converted into vinyl pyruvate as determined with NMR.

Acetylene gas in protonated or deuterated form can directly be used from suppliers. Alternatively, acetylene can be generated by the reaction of CaC₂ (calcium carbide) with water. Deuterium oxide (deuterated water) needs to be used in order to obtain deuterated acetylene.

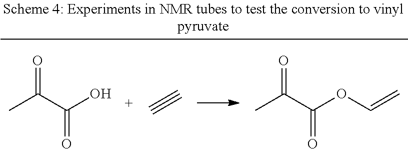

| | Reaction | C (pyru- | C (vinyl | Conversion/ |
|---|---|---|---|---|
| Solvent | time/h | vate)/mM | pyruvate)/mM | % |
| 4-Chloroanisole | 0 | 340 | 0 | 0 |
| | 42.5 | 159 | 96 | 28 |
| | 85 | 91 | 117 | 34 |
| | 149 | 27 | 166 | 49 |
| Chloroform | 0 | 398 | 0 | 0 |
| | 42.5 | 183 | 146 | 37 |
| | 85 | 77 | 214 | 54 |
| | 149 | 25 | 265 | 67 |
| Dichloroethane | 0 | 371 | 0 | 0 |
| | 42.5 | 184 | 130 | 35 |
| | 85 | 99 | 178 | 48 |
| | 149 | 21 | 240 | 65 |
| Dichloromethane | 0 | 382 | 0 | 0 |
| | 42.5 | 184 | 133 | 35 |
| | 85 | 89 | 178 | 47 |
| | 149 | 25 | 232 | 61 |
| Tetrachloroethane | 0 | 491 | 0 | 0 |
| | 42.5 | 191 | 191 | 39 |
| | 85 | 101 | 259 | 53 |
| | 149 | 14 | 325 | 66 |

### Preparation:

27 mg pyruvic acid was dissolved together with 5 mg [Ir(COD)Cl]₂ in 0.5 mL solvent in a NMR tube. The solution and tubing were degassed by pulling 5 times vacuum. Afterwards the tube was pressurized with 2.5 absolute bar acetylene gas (gas volume of 2 mL) and kept in an oil bath at 55° C. Via NMR a conversion was observed in the crude. In toluene and chlorobenzene, pyruvic acid (˜340 mM) did not dissolve completely and the reaction starts from 205 mM and 215 mM concentration respectively.

### Large Volume Reactions Including Deuterated Vinyl Pyruvate

### Large Volume Preparation of Vinyl Pyruvate:

506 mg pyruvic acid was dissolved together with 100 mg [Ir(COD)Cl]₂ in 10 mL chloroform in a 0.5 L flask. The solution and tubing was degassed by pulling 5 times vacuum. Afterwards the flask was pressurized with 1.1 absolute bar acetylene gas (gas volume of −500 mL) and kept in an oil bath at 55° C. under vigorous mixing. Via NMR a 44% conversion was observed in the crude after 6 days. After distillation 169 mg of vinyl pyruvate was obtained that corresponds to 26% yield.

### Preparation of the Deuterated Compound:

70 mg deuterated pyruvic acid was dissolved together with 20 mg [Ir(COD)Cl]₂ in 3 mL 4-chloroanisole in a 100 ml round bottom flask. The solution and tubing was degassed by pulling 5 times vacuum. Afterwards the flask was pressurized with 1 bar deuterated acetylene gas (gas volume of 100 mL) and stirred for 48 hours at 60° C. Via NMR a 40% conversion of pyruvate to vinyl pyruvate (deuterated) was observed in the crude.

Comparison with Reported Literature

The production of vinyl pyruvate (unlabelled) has been reported in the literature (Chukanov Nikita V. et al) in yields of 6%. A more recent publication which includes the advancement that vinyl pyruvate can also be produced with a 13C label (Carla Carrera et al) reports on an overall yield with respect to the pyruvate of 8%. When pyruvate is directly reacted with acetylene a yield of 26% after distillation is achieved. The conversion from pyruvate to lactate observed via NMR is 50%.

Using the deuterated vinyl pyruvate is especially useful as precursors for contrast agents. The same, most recent state of the art, as quoted above demonstrates the use of protonated vinyl pyruvate as potential contrast agent. Effectiveness of such contrast agents is reported in the literature as polarization and is usually given in percent. Thereby 0% is the lowest and 100% is the highest efficiency as dictated by considering the underlying physics. The state of the art reports on 3.2% (Chukanov Nikita V. et al) and 3.8% polarization (Carla Carrera et al) of ¹³C pyruvate which is obtained via this suitable precursor.

When using the deuterated precursor, polarization yields of 28% for pyruvate (the final contrast agent produced from deuterated vinyl pyruvate) are reported in the present invention (FIG. 1), which is nearly an ordered of magnitude better in effectiveness and is therefore an important improvement over the state of the art. Deuterated precursors for vinyl keto esters such as vinyl pyruvate have so far not been reported anywhere.

It is furthermore noteworthy that translation from a tested protonated compound to performing the same with deuterated compounds is not straight forward. So-called isotope effects have tremendous effects on reactions. This is most often encountered in a biological context (enzymatic reactions or new drugs that are not approved for clinical uses and have different properties).

### FIGURES

FIG. 1: shows the NMR spectrum of ¹³C-enhanced pyruvate obtained from the deuterated vinyl precursor (1 scan) and its comparison to the non-enhanced spectrum of the same compound which was magnified by 100 fold and measured with 200 averages. The polarization is 28%.

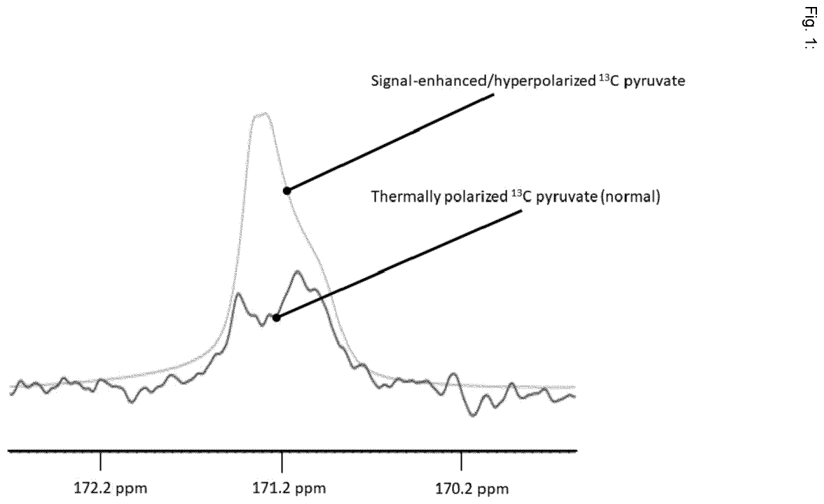

### REFERENCES

Kaltschnee et al. (2019) “Hyperpolarization of Amino Acids in Water Utilizing Parahydrogen on a Rhodium Nanocatalyst”. Chemistry. A European Journal 25 (47): 11031-11035
Michelotti et al. (2017) “Development and Scale-Up of Stereoretentive α-Deuteration of Amines”. Org. Process Res. Dev. 21 (11): 1741-1744
Saikiran et al. (2017) “Efficient near infrared fluorescence detection of elastase enzyme using peptide-bound unsymmetrical squaraine dye”. Bioorganic & Medicinal Chemistry Letters 27 (17):4024-4029
Chukanov Nikita V. et al “Synthesis of Unsaturated Precursors for Parahydrogen-Induced Polarization and Molecular Imaging of 1-13C Acetates and 1-13C-Pyruvates via Side Arm Hydrogenation”, ACS OMEGA, vol. 3, no. 6, 30 Jun. 2018, pages 6673-6682
Carla Carrera et al “ParaHydrogen Polarized Ethyl-[1-13C]pyruvate in Water, a Key Substrate for Fostering the PHIP-SAH Approach to Metabolic Imaging”, ChemPhysChem, 2021, 22, 1042-1048

1. A vinyl keto ester of formula (III), 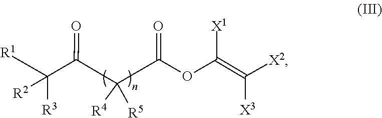
wherein
R¹, R² and R³ are independently of each other selected from H, D, and a fully or partly deuterated alkyl, particularly C₁₋₁₆-alkyl, more particularly C₁₋₆-alkyl,
each R⁴ and R⁵ are independently of any other R⁴ or R⁵ selected from H and D,
X¹, X² and X³ are independently of each other H or D, wherein at least one of X¹, X² and X³ is D, particularly all of X¹, X² and X³ are D,
n is an integer between 0 and 16, particularly between 0 and 6, more particularly between 0 and 3.

2. A method for preparing a compound suitable for signal enhanced magnetic resonance imaging comprising the steps of
a) providing a compound of formula (I), 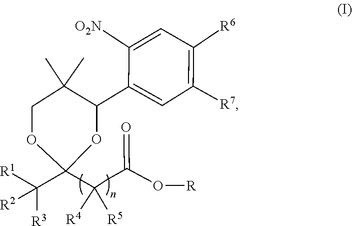
b) vinylation of the compound of formula (I) by using vinyl acetate of formula (II), 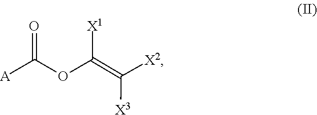
c) applying UV light yielding a vinyl keto ester of formula (III), 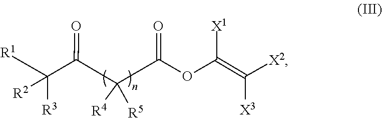
wherein
R is H or D, particularly H,
R¹, R² and R³ are independently of each other selected from H, D, and a fully or partly deuterated alkyl, particularly C₁₋₆-alkyl, more particularly C₁₋₆-alkyl, even more particularly C₁₋₃-alkyl,
each R⁴ and R⁵ are independently of any other R⁴ or R⁵ selected from H and D, particularly D,
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
R^{a} being H or a C₁₋₃-alkyl,
p being an integer between 0 and 6,
X¹, X² and X³ are independently of each other H or D, particularly D,
A is —CH₃, —CH₂D, —CHD₂ or -CD₃, particularly —CD₃, and
n is an integer between 0 and 16, particularly between 0 and 6, more particularly between 0 and 3.

3. The method according to claim 2, wherein
R¹ and R² are H or D, particularly D, and
R³ is selected from H, D and a fully or partly deuterated C₁₋₃-alkyl, particularly D, and a fully deuterated C₃-alkyl.

4. The method according to claim 2, wherein n is 0 or 1.

5. The method according to claim 2, wherein the compound of formula (I) is prepared by protecting a compound of formula (IV), 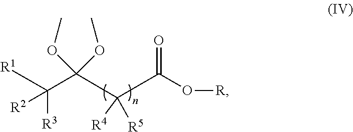
using a protecting group of formula (V), 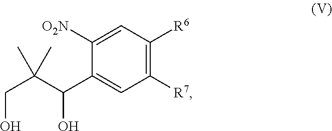
wherein R, R¹, R², R³, R⁴, R⁵, R⁶, R⁷ and n are defined as described above.

6. The method according to claim 2, wherein the vinylation in step (b) is performed repeatedly.

7. The method according to claim 2, wherein the vinyl keto ester of formula (III) is hyperpolarized after step (c), or the vinyl keto ester of formula (III) is hyperpolarized and hydrolysed after step (c).

8. A compound of formula (I) or (VI), 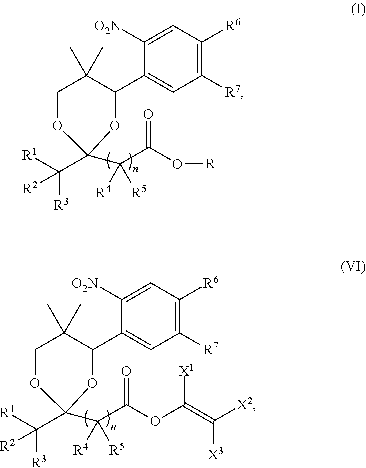
wherein
R is H or D, particularly H,
R¹, R² and R³ are independently of each other selected from H, D, and a fully or partly deuterated alkyl, particularly C₁₋₁₆-alkyl, more particularly C₁₋₆-alkyl,
each R⁴ and R⁵ are independently of any other R⁴ or R⁵ selected from H and D,
R⁶ or R⁷ is H or —OCH₃, and
the other moiety R⁷ or R⁶ is selected from H, —OCH₃, —O—CH₂—C(═O)—O—CH₂—CH₃, —CH₂—C(═O)—CH(R^{a})—NH-Boc, —O—[CH₂—CH₂—O]ₚ—H, —O—CH₂—CH(OH)—CH₂—OH, —O—CH₂—C(═O)—NH—CH₂—CH₂—NH-Boc, with
R^{a} being H or a C₁₋₃-alkyl,
p being an integer between 0 and 6,
X¹, X² and X³ are independently of each other H or D, particularly D,
n is an integer between 0 and 16, particularly between 0 and 6, more particularly between 0 and 3.

9. A method for preparing a compound suitable for signal enhanced magnetic resonance imaging comprising the steps of
a) providing a compound of formula (VII), 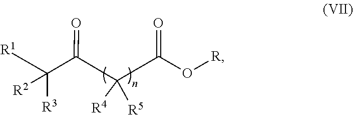
and
acetylene, wherein 0, 1 or 2 H atoms of acetylene may be replaced by D,
b) reacting the compound of formula (VII) with acetylene in the presence of a metal catalyst yielding a vinyl keto ester of formula (III), 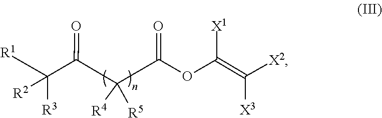
wherein
R¹, R² and R³ are independently of each other selected from H, D, and a fully or partly deuterated alkyl, particularly C₁₋₁₆-alkyl, more particularly C₁₋₆-alkyl,
each R⁴ and R⁵ are independently of any other R⁴ or R⁵ selected from H and D,
R is H or D,
X¹, X² and X³ are independently of each other H or D, particularly D, and
n is an integer between 0 and 16, particularly between 0 and 6, more particularly between 0 and 3.

10. The method according to claim 9, wherein the compound of formula (VII) is dissolved in a solvent, particularly selected from a chlorinated hydrocarbon, a chlorinated ether, a chlorinated acetophenone, acetonitrile, acetic acid, an ether, an ester, toluene, acetone, ethanol or a mix thereof, particularly from chlorinated hydrocarbon, a chlorinated ether, a chlorinated acetophenone, toluene or a mix thereof, more particularly from chlorinated hydrocarbon, a chlorinated ether, a chlorinated acetophenone or a mix thereof, wherein the solvent may optionally be fully or partly deuterated.

11. The method according to claim 9, wherein the solvent is selected from chloroform, dichloromethane, chloromethane, dichloroethane, trichloroethane, tetrachloroethane, 4′-chloroacetophenone, 4-chloroanisole and chlorobenzene, acetonitrile, acetic acid, diethyl ether, dibenzyl ether, ethyl acetate, butyl benzoate, toluene, acetone, ethanol or a mix thereof,
particularly selected from chloroform, dichloromethane, chloromethane, dichloroethane, trichloroethane, tetrachloroethane, 4′-chloroacetophenone, 4-chloroanisole and chlorobenzene,
wherein the solvent may optionally be fully or partly deuterated.

12. The method according to claim 9, wherein the metal catalyst is selected from an iridium catalyst, a rhodium catalyst, a ruthenium catalyst, a palladium catalyst, an osmium catalyst, a platinum catalyst and a rhenium catalyst, particularly an iridium(I) catalyst and a rhodium(I) catalyst.

13. The method according to claim 9, wherein the metal catalyst is selected from 1,5-cyclooctadien-iridium(I)-chloride-dimer, (1,5-cyclooctadiene) (hexafluoroacetylacetonato) iridium(I), (1,5-Cyclooctadiene)(methoxy)iridium(I) dimer, acetylacetonato(1,5-cyclooctadiene)iridium(I), dichloro(p-cymene)ruthenium(II) dimer, tris(triphenylphosphine)ruthenium(II) dichloride, [1,4-bis(diphenylphosphino)butane](1,5-cyclooctadiene)rhodium(I) tetrafluoroborate,
particularly selected from 1,5-cyclooctadien-iridium(I)-chloride-dimer, (1,5-cyclooctadiene) (hexafluoroacetylacetonato) iridium(I), acetylacetonato(1,5-cyclooctadiene)iridium(I), (1,5-Cyclooctadiene)(methoxy)iridium(I) dimer, [1,4-bis(diphenylphosphino)butane](1,5-cyclooctadiene)rhodium(I) tetrafluoroborate.

14. The method according to claim 9, wherein step (b) is performed at a temperature ≤160° C., particularly ≤60° C.

15. The method according to claim 9, wherein step (b) is performed in the presence of a polymerization inhibitor, particularly a polymerization inhibitor selected from hydroquinone, quinine and catechol.
